(19) United States
(12) Patent Application Publication
(10) Pub. No.: US 2021/0308527 A1
(43) Pub. Date: Oct. 7, 2021

(54) METHOD AND SYSTEM FOR IMPROVING QUALITY OF LIFE IN GERIATRIC AND SPECIAL NEEDS POPULATIONS

(71) Applicant: LOOK WHO'S DANCING LLC, Phoenixville, PA (US)

(72) Inventors: Helene LYNCH, Phoenixville, PA (US); Jean Anne PRINCIPATO, Blackwood, NJ (US)

(21) Appl. No.: 17/210,858

(22) Filed: Mar. 24, 2021

Related U.S. Application Data
(60) Provisional application No. 63/006,169, filed on Apr. 7, 2020.

(51) Int. Cl. A63B 24/00; G09B 19/00

(52) U.S. Cl. CPC A63B 24/0075; A63B 2244/22; G09B 19/0015

(57) ABSTRACT

Methods and kits for a dance and exercise program demonstrated to improve quality of life, as measured by interest, depression and anxiety, in geriatric and special needs populations are provided.

This patent application claims the benefit of priority from U.S. Provisional Application Ser. No. 63/006,169, filed Apr. 7, 2020, the teachings of which are incorporated herein by reference in their entirety.

### FIELD

This disclosure relates to methods and kits for a dance and exercise program demonstrated to improve quality of life, as measured by interest, depression and anxiety, in geriatric and special needs populations.

### BACKGOUND

Clinical depression in the elderly is common. That doesn't mean it's normal. Late-life depression affects about 6 million Americans ages 65 and older. But only 10% receive treatment for depression. The likely reason is that the elderly often display symptoms of depression differently. Depression in the elderly is also frequently confused with the effects of multiple illnesses and the medicines used to treat them. See https: with the extension webmd.com/depression/guide/depression-elderly#1 of the world wide web.

Anhedonia or disinterest is another important, but often overlooked, mental health concern in older adults. Research has shown that anhedonia increases with age; in fact, as many as ⅓ of older adults experience symptoms of anhedonia https: with the extension psychologybenefits.org/2018/05/31/is-losing-interest-and-motivation-inevitable-as-we-age/ of the world wide web.

Another common type of mental illness affecting people ages 60 and older is generalized anxiety disorder. See https: with the extension health.harvard.edu/mind-and-mood/treating-generalized-anxiety-disorder-in-the-elderly. Studies have also shown an increased for depression inclusive of loss of interest and anxiety in individuals with physical (See https: with the extension ncbi.nlm.nih.gov/pmc/articles/PMC5130183/of the world wide web) and intellectual disabilities (See https: with the extension ncbi.nlm.nih.gov/pmc/articles/PMC2831402/of the world wide web).

Oftentimes depression in these populations in accompanied by both anxiety and disinterest.

There are several treatment options available for depression. They include medicine, psychotherapy or counseling, or electroconvulsive therapy or other newer forms of brain stimulation (such as repetitive transcranial magnetic stimulation (rTMS)). Sometimes, a combination of these treatments may be used. See https: with the extension webmd.com/depression/guide/depression-elderly#2-5 of the world wide web.

Exercise is a behavioral intervention that has shown great promise in alleviating symptoms of depression and there is a growing body of research examining the exercise-depression relationship that supports the efficacy of exercise as an adjunct treatment. See https: with the extension ncbi.nlm.nih.gov/pmc/articles/PMC474733/of the world wide web.

Dance Movement Therapy (DMT) has been suggested as an acceptable treatment options for adults with depression in combination with accepted medicinal and/or psychotherapeutic treatments.

### SUMMARY

An aspect of the present invention relates to a method for improving quality of life, as measured by interest, depression and anxiety, in geriatric and special needs populations. The method involves an individual from a geriatric or special needs population participating in a dance/exercise program designed specifically for geriatric or special needs populations. In this dance/exercise program, dance steps are selected which provide a range of options so that any participant, regardless of age or physical or mental disability can engage in some manner. In addition, a music set list is selected to work and target specific areas or combinations of areas and to appeal to specific age groups of participants. The music set list comprises a signature opening song and a closing song. Both the selected dance steps and music set lists are divided into categories of warm-up, followed by cardio, followed by upper extremities, followed by lower extremities, followed by cool down and include means for building confidence of participants after each song in the music set list. The dance/exercise program is initiated with the signature opening song. Attention of participants is maintained through individual engagement via one or more dance/exercise program instructors greeting each participant and caregiver by name, making eye contact with each participant, when accepted shaking hands or gently touching each participant in a friendly manner, and calling out encouraging words to each participant throughout the session. Participants are continually verbally reminded of each dance step throughout every class or session of the dance/exercise program regardless of whether the participants are familiar with the dance steps. Music selections are modified and/or short breaks are provided in accordance with energy level of the participants. Each session or class of the dance/exercise program ends with the selected closing signature song and all participants are encouraged to take a final bow together as a class.

Another aspect of the present invention relates to a training kit for individuals interested in implementing methods for improving quality of life, as measured by interest, depression and anxiety, in geriatric and special needs populations. The kit comprises a manual outlining a dance/exercise program designed specifically for geriatric or special needs populations, a video of one or more sessions of the dance/exercise program in progress demonstrating each of the actions and steps described in the manual for reference by the instructor(s) to ensure that the program is being performed as instructed to provide improvement in the quality of life of the participants, and one or more music set lists with dance steps for the dance/exercise program each inclusive of warm-up, followed by cardio, followed by upper extremities, followed by lower extremities, followed by cool down as well as signature opening and closing songs.

### DETAILED DESCRIPTION

The present invention provides a method and kit for improving quality of life, as measured by interest, depression and anxiety, in geriatric and special needs populations.

In the method of the present invention, individuals from geriatric and special needs population participate in a dance/exercise program designed specifically for geriatric or special needs populations.

This dance/exercise program comprises selecting dance steps which provide a range of options so that any participant, regardless of age or physical or mental disability can engage in some manner. As a nonlimiting example, the choreographed move may be for a participant to kick their feet. For participants confined to wheel chair, the instructor or an assistant may demonstrate a modified move using arms to simulate a kick or (if able) any foot movement to the extent that their ability allows. Focus is on what the participant can do and every participant's engagement level in the dance/exercise program may be different. For example, some students will be able to complete every move in the program, while others may participate on the periphery wandering around the room or dancing their own special dance. Participants with severe physical challenges may not be able to physically participate, but can participate in social aspects including listening to the music and watching other participants and the instructor. All participants are encouraged to do what they are capable of doing, despite any hindrances, whether in a chair, using a walker, or sitting on the couch.

The dance/exercise program of the present invention further comprises selecting and playing music. Music is selected to work and target specific areas or combinations of areas. Different songs are selected to promote use of arms, legs, and core. As a nonlimiting example, the song “Footloose” is selected and played to target legs and/or core.

Further, music is modified based upon specific age groups of participants. As a nonlimiting example, for a geriatric class it has been found that music from their era, such as songs by Frank Sinatra promote memory. As another nonlimiting example, for a special needs class made up of younger participants, clean versions of popular songs may be selected.

Music set lists for the dance/exercise program are provided to instructors of the dance exercise program. Signature opening and closing music selections are provided for all classes. It has been found that use the same opening, warm-up songs at all classes helps participants focus and stimulates muscle memory for the opening of the program. Many participants will get to know the opening songs and start to clap even before the instructor begins. Closing songs, typically two, are also the same for each program as it signals that class will be ending, and allows a time for cool down.

Dance steps and music selections are divided into the following categories of warm-up, followed by cardio, followed by upper extremities, followed by lower extremities, followed by cool down.

This dance/exercise program of the present invention further comprises maintaining the attention of each participant throughout the program. Examples for maintaining a participant's attention include individual engagement via one or more instructors greeting each participant and caregiver by name, making eye contact with each participant, when accepted shaking hands or gently touching each participant in a friendly manner, and calling out encouraging words to each participant throughout the session. Attention of each participant is also maintained by arranging participants in a format where they can easily see the instructor. In a preferred embodiment, participants are arranged in a large circle with one or more instructors in the center. In the event a circle format is not an option, instructors must face the participants. When arranging participants, those sensitive to loud noises should be placed at the beginning of the session away from any speakers. Alternatively, or in addition, participants sensitive to loud noises are encouraged to wear ear/sound blocking apparatus.

The dance/exercise program of the present invention further comprises instructors continually reminding the participants of each dance step throughout the class or session regardless of whether the participants are familiar with the choreography. With each session, participants may learn the choreography. However, the inventors have found that participants are more comfortable with continual reminders of every dance move being called out verbally during each session.

The dance/exercise program of the present invention further comprises modifying the music selections and/or providing breaks in accordance with the energy level of the participants. For example, there is typically a higher energy level at the beginning of the class or session which starts to decrease about 15 to 20 minutes into the class or session. Accordingly, faster music selections are typically played at the beginning of class while slower selections of music are played as participants' energy levels begin to drop. One or more short water breaks of 2 minutes or less thus preventing loss of attention may also be incorporated into the session. The only time the music stops is during the brief water break.

Sessions run from 35 to 45 minutes depending upon the endurance of the group of participants.

The dance/exercise program further comprises incorporating a means for building confidence of participants after each song in the music list. Such means include applause, thumbs up, high fives to all participants, taking bows, individual recognition of participants by name and words of encouragement from the instructor such as, but not limited to, “Good job” “Well done” and “You are awesome”. In some embodiments, bows are built into the choreography.

The dance/exercise program further comprises ending the session or class with the selected signature song and encouraging all participants to raise both hands when possible and take a final bow together as a class, thus allowing participants to feel like part of a group. Following this final bow, the dance/exercise program further comprises instructors providing words of encouragement and giving thanks to each participant and caregiver by name for participating in the class or session.

The dance/exercise program further comprises introducing new music and dances very slowly as routine is a very important aspect of this invention. After several weeks or even months, a single song in the music list may be changed and then this same order of music with the single change is repeated for the next several weeks to months. If the change is not well-received by the majority of participants, return to the prior routine may be needed.

In a preferred embodiment, the dance/exercise program, is provided in person, as in person classes provide an excellent format for instructors to engage with and encourage each student to participate to their abilities, as well as a means for social interaction between participants.

However, it has also been found that the dance/exercise program can be effectively performed in a virtual format, with students still being able to engage with their instructors and to interact socially with all participants via their computer or other electronic device. This virtual format provides for expansion of this program to individuals who can benefit from the program but may not have means or ability to attend an in person class.

The present invention also provides kits to be provided to instructors for training in this dance/exercise program. The kit comprises a written manual outlining the dance/exercise program. The kit further comprises a video of one or more sessions of the dance/exercise program in progress demonstrating each of the actions and steps described in the manual for reference by the instructor(s) to ensure that the program is being performed as instructed to provide improvement in the quality of life of the participants. In addition, the kits of the present invention comprise one or more music set lists with dance steps for the dance/exercise program each inclusive of warm-up, followed by cardio, followed by upper extremities, followed by lower extremities, followed by cool down as well as signature opening and closing songs.

This dance/exercise program has been described by participants or their caregivers as not only improving the physical wellbeing of participants but also improves their mental/emotional health and self-esteem. Unlike with schooling and therapies, no one is correcting or attempting to “normalize” participants. Sessions or classes have been found to exhibit 95% participation. Further, individuals who do not participate in other activities have been shown to participate and enjoy these classes or session and/or engage in the class from start to finish without loss of interest.

The following nonlimiting example is provided to further illustrate the present invention.

### EXAMPLE

Studies were conducted by researchers at University of Pennsylvania School of Nursing. Forty participants in a memory unit at a skilled nursing facility were divided into 2 groups based on age, gender, health risks, and ability levels. Both groups were seniors who were mostly confined to wheelchairs. Group 1 took the class every week for 12 weeks. Group 2 took the class every other week for twelve weeks, total of 6 classes. Members of the staff at the nursing facility were instructed in the use of the “Apparent Affect Rating Scale” (AARS).

Statistically significant improvements in measures of participants'sadness and interest in daily like were found in both groups. Borderline decreases in anxiety were also observed.

What is claimed:

1. A method for improving quality of life in geriatric and special needs populations, said method comprising involving an individual from a geriatric or special needs population participating in classes or sessions of a dance/exercise program, said dance exercise program comprising:
(a) dance steps which provide a range of options so that any participant, regardless of age or physical or mental disability can engage in some manner;
(b) a music set list selected to work and target specific areas or combinations of areas and to appeal to specific age groups of participants, said music set list comprising a signature opening song and a closing song;
wherein said dance steps and music set list are divided into categories of warm-up, followed by cardio, followed by upper extremities, followed by lower extremities, followed by cool down;
(c) initiating the dance/exercise program with the signature opening song;
(d) maintaining a participant's attention in the dance/exercise program through individual engagement via one or more dance/exercise program instructors greeting each participant and caregiver by name, making eye contact with each participant, when accepted shaking hands or gently touching each participant in a friendly manner, and calling out encouraging words to each participant throughout the session;
(e) continually verbally reminding the participants of each dance step throughout every class or session of the dance/exercise program regardless of whether the participants are familiar with the dance steps;
(f) modifying music selections and/or providing breaks in accordance with energy level of the participants;
(g) incorporating a means for building confidence of participants after each song in the music set list;
(h) ending the session or class with the selected closing signature song;
(i) encouraging all participants to take a final bow together as a class.

2. The method of claim 1 wherein improvement in quality of life is assessed by increased interest and/or decreased depression and/or decreased anxiety in the individual.

3. The method of claim 1 wherein attention of each participant is further maintained by arranging participants in a format where they can easily see the instructor.

4. The method of claim 3 wherein the format is a large circle with one or more instructors in the center.

5. The method of claim 3 wherein the format is a virtual class.

6. The method of claim 1 wherein participants sensitive to loud noises are placed away from any speakers at the beginning of the session.

7. The method of claim 1 wherein participants sensitive to loud noises wear ear/sound blocking apparatus.

8. A kit comprising:
a written manual outlining the dance/exercise program of claim 1;
a video of one or more sessions of the dance/exercise program in progress demonstrating each of the actions and steps described in the manual for reference by an instructor(s) to ensure that the program is being performed as instructed to provide improvement in the quality of life of the participants; and
one or more music set lists with dance steps for the dance/exercise program each inclusive of warm-up, followed by cardio, followed by upper extremities, followed by lower extremities, followed by cool down as well as signature opening and closing songs.
